(19) United States
(12) Patent Application Publication
(10) Pub. No.: US 2025/0273062 A1
(43) Pub. Date: Aug. 28, 2025

(54) PREDICTIVE PORTABLE DEVICE

(71) Applicant: SAUDI ARABIAN OIL COMPANY, Dhahran (SA)

(72) Inventors: Basil Abuhadi, Jeddah (SA); Francisco V. Ramos, Dhahran (SA); Mohammed A. Alswayied, Riyadh (SA)

(73) Assignee: SAUDI ARABIAN OIL COMPANY, Dhahran (SA)

(21) Appl. No.: 18/589,881

(22) Filed: Feb. 28, 2024

(51) Int. Cl. G08B 21/14; G01N 33/00

(52) U.S. Cl. CPC G08B 21/14; G01N 33/0044; G01N 33/0065; G01N 33/0075

(57) ABSTRACT

A system includes first and second sensors configured to periodically measure a concentration of a noxious gas and a level of ambient noise, respectively. A first control/monitoring unit is configured to receive periodic measurements from the first and second sensors. The first control/monitoring unit is further configured to: calculate a cumulative total exposure to the noxious gas and ambient noise; calculate a remaining permissible exposure time for the noxious gas and ambient noise; and, based on the calculation of the remaining permissible exposure times, transmit a signal to a first alarm to activate the same. A related method includes: periodically measuring a concentration of a noxious gas and a level of ambient noise and calculating a cumulative total exposure to both. A remaining permissible exposure time is calculated for each of the noxious gas and the ambient noise, on the basis of which a first alarm is activated.

### BACKGROUND

In many industrial facilities, and particularly in certain portions of such facilities, operators may be exposed to a variety of hazards over extended periods of time, and guidelines are typically in place to limit such exposure. For instance, in oil and gas production, operators may be exposed to noxious gases such hydrogen sulfide (H2S), and to significant levels of noise, in different locations that may include shipping pumps, gas turbine generators or compressors.

Precision instrumentation may be able to gauge an operator's accumulated exposure to an individual hazard over a given period of time. However, it remains a challenge to assess the combined effect of disparate hazards in a given environment over a given period of time.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In one aspect, embodiments disclosed herein relate to a system that includes a first sensor configured to periodically measure a concentration of a noxious gas and a second sensor configured to periodically measure a level of ambient noise. A first control/monitoring unit is in communication with the first and second sensors, and is configured to receive from the first and second sensors periodic measurements of the concentration of the noxious gas and the ambient noise level. A first alarm is in communication with the first control/monitoring unit. The first control/monitoring unit is further configured to, based on the received periodic measurements, calculate a cumulative total exposure to each of: the noxious gas; and the ambient noise. Based on the calculation of the cumulative total exposures, the first control/monitoring unit is configured to calculate a remaining permissible exposure time for each of: the noxious gas; and the ambient noise. Based on the calculation of the remaining permissible exposure times, the first control/monitoring unit is configured to transmit a signal to the first alarm to activate the first alarm.

In one aspect, embodiments disclosed herein relate to a method that includes: periodically measuring a concentration of a noxious gas, with a first sensor; periodically measuring a level of ambient noise, with a second sensor; and receiving from the first and second sensors periodic measurements of the concentration of the noxious gas and the ambient noise level. Based on the received periodic measurements, there is calculated a cumulative total exposure to each of: the noxious gas; and the ambient noise. Based on the calculation of the cumulative total exposures, there is calculated a remaining permissible exposure time for each of: the noxious gas; and the ambient noise. Based on the calculation of the remaining permissible exposure times, a signal is transmitted to a first alarm to activate the first alarm.

Other aspects and advantages of the claimed subject matter will be apparent from the following description and the appended claims.

### BRIEF DESCRIPTION OF DRAWINGS

Specific embodiments of the disclosed technology will now be described in detail with reference to the accompanying figures. Like elements in the various figures are denoted by like reference numerals for consistency.

FIG. 1 schematically illustrates, in a cross-sectional elevational view, a wellbore and a well control system in accordance with one or more embodiments.

FIG. 2 schematically illustrates a hazardous venue and related components, in accordance with one or more embodiments.

FIG. 3 shows a flowchart of a method in accordance with one or more embodiments.

FIG. 4 schematically illustrates a computing device and related components, in accordance with one or more embodiments.

### DETAILED DESCRIPTION

In the following detailed description of embodiments of the disclosure, numerous specific details are set forth in order to provide a more thorough understanding of the disclosure. However, it will be apparent to one of ordinary skill in the art that the disclosure may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements nor to limit any element to being only a single element unless expressly disclosed, such as using the terms “before”, “after”, “single”, and other such terminology. Rather, the use of ordinal numbers is to distinguish between the elements. By way of an example, a first element is distinct from a second element, and the first element may encompass more than one element and succeed (or precede) the second element in an ordering of elements.

Broadly contemplated herein, in accordance with one or more embodiments, are systems and methods for undertaking measurements of H2S (or other noxious gas) concentrations and noise levels over a variety of exposure time durations, and gauging the frequency and intensity of an operator's exposure to such hazards over time. More particularly, if accumulated exposure to the noted hazards reaches a predetermined threshold, such as a margin prior to a maximum permissible exposure time (based on one or more predetermined standards or recommended limits), then a first alarm may be generated (e.g., to alert one or more operators to leave the area). Further, if the noted hazards are measured to exceed the recommended limits, then another alarm may be generated (e.g., to alert another individual such as a supervisor).

In accordance with one or more embodiments, sensors for measuring H2S (or other noxious gas) concentrations and noise levels are combined into one system that incorporates one or more algorithms or methods for computing permissible exposure time based on predetermined standards. The system may be embodied by a portable unit carried or worn by an operator.

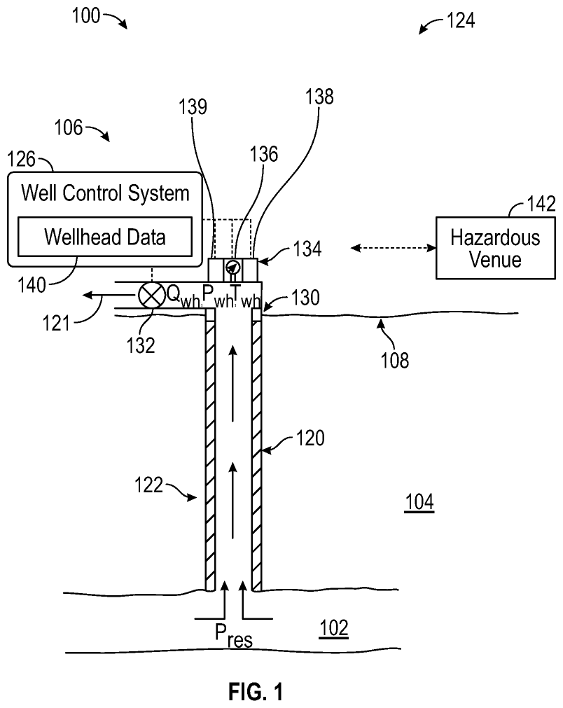

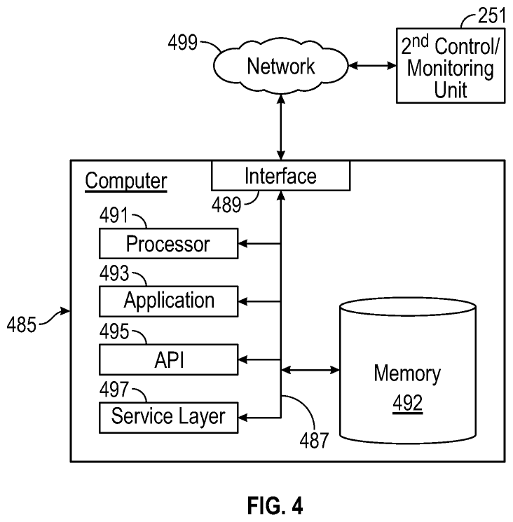

In accordance with one or more embodiments, FIG. 1 illustrates a general environment in which one or more embodiments may be employed. Thus, FIG. 1 schematically illustrates, in a cross-sectional elevational view, a wellbore and a well control system in accordance with one or more embodiments. The well system 106 includes a wellbore 120, a well sub-surface system 122, a well surface system 124, and a well control system (“control system”) 126. The control system 126 may control various operations of the well system 106, such as well production operations, well completion operations, well maintenance operations, and reservoir monitoring, assessment and development operations. The control system 126 includes a computer system that can be the same as, or is in communication with, computer system 485 described below in FIG. 4.

In accordance with one or more embodiments, the wellbore 120 includes a bored hole that extends from the surface 108 into a target zone of the formation 104, such as the reservoir 102. An upper end of the wellbore 120, terminating at or near the surface 108, may be referred to as the “up-hole” end of the wellbore 120, and a lower end of the wellbore, terminating in the formation 104, may be referred to as the “down-hole” end of the wellbore 120. The wellbore 120 facilitates the circulation of drilling fluids during drilling operations, the flow of hydrocarbon production (“production”) 121 (e.g., oil and gas) from the reservoir 102 to the surface 108 during production operations, the injection of substances (e.g., water) into the formation 104 or the reservoir 102 during injection operations, or the communication of monitoring devices (e.g., logging tools) into the formation 104 or the reservoir 102 during monitoring operations (e.g., during in situ logging operations).

In accordance with one or more embodiments, during operation of the well system 106, the control system 126 collects and records wellhead data 140 for the well system 106. The wellhead data 140 may include, for example, a record of measurements of wellhead pressure (P_{wh}) (e.g., including flowing wellhead pressure), wellhead temperature (T_{wh}) (e.g., including flowing wellhead temperature), wellhead production rate (Q_{wh}) over some or all of the life of the well 106, and water cut data. Such measurements may be recorded in real-time, to be available for review or use within seconds, minutes, or hours of the condition being sensed (e.g., within one hour). Such real-time data can help an operator of the well 106 to assess a relatively current state of the well system 106, and make real-time decisions regarding development of the well system 106 and the reservoir 102, such as on-demand adjustments in regulation of production flow from the well.

In accordance with one or more embodiments, the well sub-surface system 122 includes a casing installed in the wellbore 120. For example, the wellbore 120 may have a cased portion and an uncased (or “open-hole”) portion. The cased portion may include a portion of the wellbore having casing disposed therein. The uncased portion may include a portion of the wellbore not having casing disposed therein. In embodiments having a casing, the casing defines a central passage that provides a conduit for the transport of tools and substances through the wellbore 120. For example, the central passage may provide a conduit for lowering logging tools into the wellbore 120, a conduit for the flow of production 121 (e.g., oil and gas) from the reservoir 102 to the surface 108, or a conduit for the flow of injection substances (e.g., water) from the surface 108 into the formation 104. The well sub-surface system 122 can include production tubing installed in the wellbore 120. The production tubing may provide a conduit for the transport of tools and substances through the wellbore 120. The production tubing may, for example, be disposed inside casing. In such an embodiment, the production tubing may provide a conduit for some or all of the production 121 (e.g., oil and gas) passing through the wellbore 120 and the casing.

In accordance with one or more embodiments, the well surface system 124 includes a wellhead 130. The wellhead 130 may include a rigid structure installed at the “up-hole” end of the wellbore 120, at or near where the wellbore 120 terminates at the Earth's surface 108. The wellhead 130 may include structures (called “wellhead casing hanger” for casing and “tubing hanger” for production tubing) for supporting (or “hanging”) casing and production tubing extending into the wellbore 120. Production 121 may flow through the wellhead 130, after exiting the wellbore 120 and the well sub-surface system 122, including, for example, the casing and the production tubing. The well surface system 124 may include flow regulating devices that are operable to control the flow of substances into and out of the wellbore 120. For example, the well surface system 124 may include one or more production valves 132 that are operable to control the flow of production 121. For instance, a production valve 132 may be fully opened to enable unrestricted flow of production 121 from the wellbore 120, the production valve 132 may be partially opened to partially restrict (or “throttle”) the flow of production 121 from the wellbore 120, and production valve 132 may be fully closed to fully restrict (or “block”) the flow of production 121 from the wellbore 120, and through the well surface system 124.

In accordance with one or more embodiments, the wellhead 130 includes a choke assembly. For example, the choke assembly may include hardware with functionality for opening and closing the fluid flow through pipes in the well system 106. Likewise, the choke assembly may include a pipe manifold that may lower the pressure of fluid traversing the wellhead. As such, the choke assembly may include set of high pressure valves and at least two chokes. These chokes may be fixed or adjustable or a mix of both. Redundancy may be provided so that if one choke has to be taken out of service, the flow can be directed through another choke. In some embodiments, pressure valves and chokes are communicatively coupled to the well control system 126. Accordingly, a well control system 126 may obtain wellhead data regarding the choke assembly as well as transmit one or more commands to components within the choke assembly in order to adjust one or more choke assembly parameters.

In accordance with one or more embodiments, the well surface system 124 includes a surface sensing system 134. The surface sensing system 134 may include sensors for sensing characteristics of substances, including production 121, passing through or otherwise located in the well surface system 124. The characteristics may include, for example, pressure, temperature and flow rate of production 121 flowing through the wellhead 130, or other conduits of the well surface system 124, after exiting the wellbore 120.

In accordance with one or more embodiments, the surface sensing system 134 includes a surface pressure sensor 136 operable to sense the pressure of production 121 flowing through the well surface system 124, after it exits the wellbore 120. The surface pressure sensor 136 may include, for example, a wellhead pressure sensor that senses a pressure of production 121 flowing through or otherwise located in the wellhead 130. In some embodiments, the surface sensing system 134 includes a surface temperature sensor 138 operable to sense the temperature of production 121 flowing through the well surface system 124, after it exits the wellbore 120. The surface temperature sensor 138 may include, for example, a wellhead temperature sensor that senses a temperature of production 121 flowing through or otherwise located in the wellhead 130, referred to as “wellhead temperature” (T_{wh}). In some embodiments, the surface sensing system 134 includes a flow rate sensor 139 operable to sense the flow rate of production 121 flowing through the well surface system 124, after it exits the wellbore 120. The flow rate sensor 139 may include hardware that senses a flow rate of production 121 (Q_{wh}) passing through the wellhead 130.

In accordance with one or more embodiments, the well system 106 may be an oil or gas well system and the reservoir 102 may be an oil or gas reservoir. Additionally, and as noted heretofore, a hazardous venue 142 may be present where operators may be exposed to noxious gases such as H2S, and to significant levels of noise. Such a venue 142 may be an enclosed space, above-ground or underground, that may, merely by way of illustrative example, incorporate—or be located in the vicinity of—shipping pumps, gas turbine generators or compressors. Generally, the hazardous venue 142 may be embodied by essentially any enclosed or open space at or near a well system 106, or at any of a great variety of other industrial facilities, where operators may be exposed to a noxious gas and to significant levels of noise.

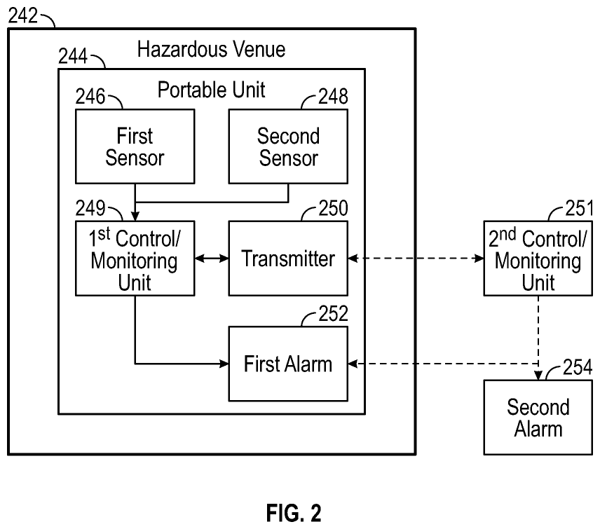

The disclosure now turns to working examples of a system and method in accordance with one or more embodiments, as described and illustrated with respect to FIGS. 2-4. It should be understood and appreciated that these merely represent illustrative examples, and that a great variety of possible implementations are conceivable within the scope of embodiments as broadly contemplated herein.

FIG. 2 schematically illustrates a hazardous venue 242 and a portable sensing unit 244, in accordance with one or more embodiments. The hazardous venue 242 may be analogous to that (142) described and illustrated with respect to FIG. 1, or may represent another hazardous venue where operators may be exposed to one or more noxious gases such as H2S, and to significant levels of noise. As such, an operator may enter the hazardous venue 242 as noted heretofore and may carry the portable sensing unit 244 essentially in any suitable manner. Thus, the portable sensing unit 244 may be in the form of a self-contained device, e.g., in the shape of a rectilinear box, that the operator can wear or carry on a lanyard or belt. Other possible modes of carry for the portable sensing unit 244 may include, but certainly need not be limited to, wearing, clipping or mounting the unit 244 on clothing (e.g., at or near a collar, lapel, or breast) or on a helmet.

In accordance with one or more embodiments, within the portable sensing unit 244, a first sensor 246 is configured to measure a concentration of a noxious gas (such as H2S) and a second sensor 248 is configured to measure an ambient noise level. Each of the two sensors 246, 248 may be configured to take their respective measurements at predetermined time intervals, such as once per second. Merely by way of illustrative and non-restrictive example, first sensor 246 may be embodied by an electrochemical H2S sensor and second sensor 248 may be embodied by an integrated microphone, microphone array or pressure sensor. Essentially any suitable sensor or sensing arrangement may be employed for first and second sensors 246, 248, wherein a sensing end or sensing portion of each may be sufficiently exposed to ambient in a manner to obtain sufficiently reliable readings.

In accordance with one or more embodiments, while H2S is discussed herein as one possible noxious gas to which an operator may be exposed, and sensed by first sensor 246, it should be understood that first sensor 246 may alternatively sense, and calculate an operator's exposure to, any of a great variety of other noxious gases such as carbon dioxide or carbon monoxide. The first sensor 246 may alternatively be used as a LEL (lower explosive limit) sensor, toward determining a lowest concentration of gas mixed with air that can ignite.

In accordance with one or more embodiments, the periodic measurements taken by the first and second sensors 246/248 are transmitted to a first control/monitoring unit 249 that resides in the portable unit 244. In this respect, the first control/monitoring unit 249 may be embodied by a CPU (central processing unit) or other internal logic suitable for carrying out functions described herein. Additionally, the periodic measurements taken by the first and second sensors 246/248 may be transmitted, via a suitable transmitter 250, to a second control/monitoring unit 251.

The second control/monitoring unit 251 may be at a location that is remote from the hazardous venue 242 and may, for instance, reside in a general control panel located in a building configured for general control purposes. The second control/monitoring unit 251 may embodied similarly to the first control/monitoring unit 249, to carry out functions as described herein, though may be of a larger physical scale if deemed suitable. The second control/monitoring unit 251, alternatively, may form part of a well control system such as that indicated at 126 in FIG. 1, or may even be located at or within the hazardous venue 242. The transmitter 250 may embodied in essentially any manner deemed suitable in the context of transmitting data from first and second sensors 246/248. For instance, merely by way illustrative and non-restrictive example, transmitter 250 may be embodied to utilize 4-20 mA (milliampere) HART protocol, “MODBUS” protocol or essentially any method that supports data encryption. (“MODBUS” is a registered trademark of Schneider Electric USA, Inc.)

Generally, in accordance with one or more embodiments, the second control/monitoring unit 251 may be in communication with a computer 485 such as that described and illustrated with respect to FIG. 4. Such communication may be utilized for further analysis of the data transmitted from first and second sensors 246/248. Alternatively, the second control/monitoring unit 251 may form part of the computer 485 itself, e.g. as a constituent portion of an application 493.

In accordance with one or more embodiments, the first control/monitoring units 249 may be configured to receive the periodic measurements from sensors 246/248 and also, based on the periodic measurements, to calculate a cumulative total exposure of the operator to the noxious gas and the ambient noise. Such calculations may be rendered at predetermined intervals, such as once per second. In accordance with at least one variant, the second control/monitoring unit 251 may render similar calculations, instead of, in collaboration with or in addition to the first control/monitoring unit 249.

In accordance with one or more embodiments, the first control/monitoring unit 249 may calculate a remaining permissible exposure time to each of (a) the noxious gas and (b) the ambient noise. This may be accomplished in essentially any suitable manner, e.g., via a linear or logarithmic prediction or extrapolation. In accordance with at least one variant, the second control/monitoring unit 251 may render similar calculations, instead of, in collaboration with or in addition to the first control/monitoring unit 249.

In accordance with one or more embodiments, a remaining permissible exposure time to each of (a) the noxious gas and (b) the ambient noise can first be understood as a function of a maximum permissible exposure time. The maximum permissible exposure time for each hazard (noxious gas or ambient noise) may conceivably be a static quantity if the hazard is present in the venue 242 at a constant level on a continual basis, for the duration of the operator's presence in the venue 242. In practice, the maximum permissible exposure time for each hazard (noxious gas or ambient noise) may well be a dynamic quantity that varies based on varying exposure to differing levels of each respective hazard at different times. Thus, the first sensor 246 may well detect varying levels of a noxious gas at different physical locations at the hazardous venue 242. The second sensor 248, likewise, may detect varying noise levels at different physical locations at the hazardous venue 242 but may also be subject to a variety of noise sources, of differing intensity, at one and the same physical location.

Thus, in accordance with one or more embodiments, as measurements are taken by the first and second sensors 246/248 during a period of time that an operator is present in the hazardous venue 242, any of a variety of viable methods of linear or logarithmic prediction or extrapolation may be utilized to generate a best estimate of the maximum permissible exposure time for each hazard. The remaining permissible exposure time for each hazard may then be determined as the time remaining until a current best estimate of the maximum permissible exposure time.

In accordance with one or more embodiments, ambient noise measurements taken over a given period of time via second sensor 248, e.g., between the beginning of an operator's shift and a current timepoint, may be added and then averaged, and a current average value—or “cumulative average”—may then be used as a basis for determining a maximum permissible exposure time. Thus, this maximum permissible exposure time may then be used for determining a remaining permissible noise exposure time, and can continue to vary based on new, incoming noise readings via second sensor 248. At the same time, measurements of noxious gas taken over a given period of time via first sensor 246 may be handled in a more linear fashion. Particularly, a remaining permissible exposure time to noxious gas may be determined as a function of the total cumulative exposure to the measured noxious gas. Generally, it should be understood that a remaining permissible exposure time for either or both of a noxious gas and ambient noise may be calculated in essentially any manner deemed suitable, e.g., via a pre-programmed plot or lookup table incorporated by first control/monitoring unit 249, and in a manner that may be linear or non-linear.

In accordance with one or more embodiments, based on the calculation of remaining permissible exposure times as noted, the operator may be alerted, via an alarm, when one or more predetermined thresholds has been reached for either or both of (a) the noxious gas and (b) the ambient noise. Thus, a first alarm 252 may be included as a constituent portion of the portable sensing unit 244 and may be in communication with the first control/monitoring unit 249. Thus, once one or more of the thresholds has been reached, the first control/monitoring unit 249 may send a signal to first alarm 252 in order to activate the same and to alert the operator. As an alternative, an alarm functioning in similar manner, instead of or in addition to the first alarm 252, may be disposed at a fixed location at the hazardous venue (e.g., as a wall-mounted unit), for the purpose of alerting the operator. As another alternative, the second control/monitoring unit 251 may send a signal to activate the first alarm 252 instead of instead of, in collaboration with or in addition to the first control/monitoring unit 249. The first alarm 252 may be an auditory alarm, to create one or more distinct sounds upon activation, or a visual alarm, to create one or more visual patterns (e.g., a flashing light) upon activation, or may include both auditory and visual features.

In accordance with one or more embodiments, the first control/monitoring unit 249 transmits a signal to first alarm 252, to cause the first alarm 252 to activate and to alert the operator, when either or both of the following predetermined thresholds has been reached for either or both of (a) the noxious gas and (b) the ambient noise:
A predetermined discrete time interval prior to the maximum permissible exposure time is reached (e.g., 10 minutes prior).
A predetermined proportion of the maximum permissible exposure time, and less than 100% thereof (e.g., 90% of the total), has elapsed (i.e., is reached).

Additionally, in accordance with one or more embodiments, a second alarm 254, functioning similarly to first alarm 252, may be disposed at another location that is remote from the hazardous venue 242. Thus, for instance, the second alarm 254 may be disposed in a room or building where another individual, such as a supervisor of the operator, is located. Similarly to the first alarm 252, the second alarm 254 may also be a constituent portion of a portable unit or may be in a fixed location (e.g., wall-mounted) at the remote venue. By way of an illustrative and non-restrictive example, the second alarm 254 may be disposed in a building or room where the second control/monitoring unit 251 is also located. As such, the second alarm 254 may be activated under the same conditions as noted above for the first alarm 252. To that end, the first control/monitoring unit 249 may send a signal via transmitter 250 to the second control/monitoring unit 251, that itself may send a signal to second alarm 254 and thereby cause the second alarm 254 to activate. In the process, the second control/monitoring unit 249 may send a signal back to the transmitter 250 and first control/monitoring unit 249 as an acknowledgement. In accordance with at least one variant, the transmitter 250 may send an activation signal directly to the second alarm 254 instead of to the second control/monitoring unit 251.

In accordance with one or more embodiments, the activation of first and/or second alarms 252/254, as noted above, may be regarded as a “first activation.” As such, a subsequent “second activation” of either or both of first alarm 252 and second alarm 254 may also take place when the maximum permissible exposure time has actually been reached for either or both of (a) the noxious gas and (b) the ambient noise.

In accordance with one or more embodiments, a very wide variety of possibilities exist for the first activation and second activation processes noted above. For instance, different sounds, tones or patterns may be provided for activating the first and/or second alarms 252/254 in connection with the first activation process, such that a first sound, tone, or pattern signifies the predetermined threshold for the noxious gas and a second sound, tone auditory pattern, or visual pattern—distinct from the first sound, tone, auditory pattern, or visual pattern—signifies the predetermined noise threshold. Additionally, different sounds, tones or patterns may be provided for activating the first and/or second alarms 252/254 in connection with the second activation process, such that a third sound, tone, auditory pattern, or visual pattern signifies the maximum permissible exposure time for the noxious gas and a fourth sound, tone, or pattern—distinct from the third sound, tone, auditory pattern, or visual pattern—signifies the maximum permissible exposure time for noise. Further, the third sound, tone, auditory pattern or visual pattern may be distinct from or the same as the first sound, tone, auditory pattern, or visual pattern, while the fourth sound, tone, auditory pattern, or visual pattern may be distinct from or the same as the second sound, tone, auditory pattern, or visual pattern.

In accordance with one or more embodiments, for either or both of the first activation and second activation processes noted above, the first control/monitoring unit 249 and/or second control/monitoring unit 251 can execute a voting process that determines a safest or most conservative threshold. For instance, for the first activation process, a voting process can determine which of the following four thresholds will be reached earliest: the predetermined discrete time interval, for each of (a) the noxious gas and (b) the ambient noise; and the predetermined proportion of the maximum permissible exposure time, for each of (a) the noxious gas and (b) the ambient noise. For the second activation process, a voting process can determine whether the maximum permissible exposure time will be reached earlier for (a) the noxious gas or (b) the ambient noise. For each of the first and second activation processes, the first and/or second control/monitoring units 249/251 may then send an activation signal to either or both of the first and second alarms 252/254 based on the threshold that is reached earliest (for the first activation process) and on the maximum permissible exposure time that is reached earlier (for the second activation process).

Generally, in accordance with one or more embodiments, the first control/monitoring unit 249 is configured to determine at least two thresholds, based on the calculation of the remaining permissible exposure times, for each of (a) the noxious gas and (b) the ambient noise. Thus, the first control/monitoring unit 249 may determine the thresholds discussed above but may alternatively determine, for each of (a) the noxious gas and (b) the ambient noise, one or more additional thresholds or—generally speaking—two or more thresholds that need not necessarily include the thresholds discussed above. From among the determined thresholds, at least four total in number, the first control/monitoring unit 249 may then transmit the signal to first alarm 252 and/or second alarm 254 when an earliest one of the determined thresholds is reached. The earliest one of the determined thresholds may thus itself be determined via a voting process of the type discussed above. In accordance with at least one variant, the second control/monitoring unit 251 may act to determine thresholds as noted instead of, in collaboration with or in addition to the first control/monitoring unit 249.

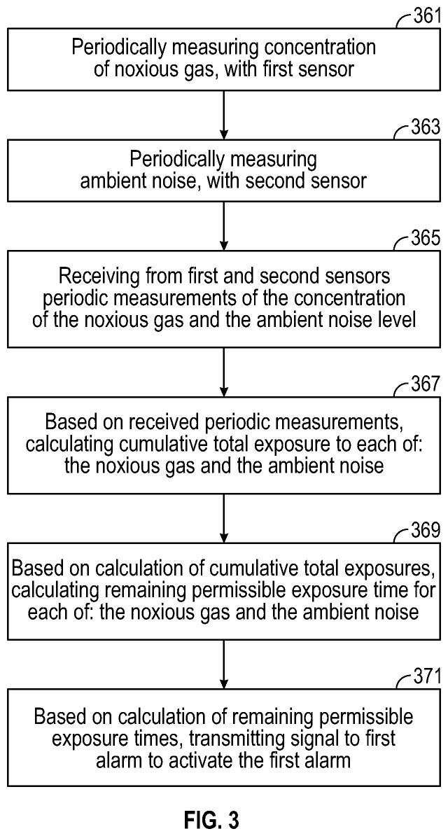

FIG. 3 shows a flowchart of a method, as a general overview of steps which may be carried out in accordance with one or more embodiments described or contemplated herein. Specifically, FIG. 3 describes a method of activating an alarm based on measured concentrations of a noxious gas and measured levels of ambient noise. One or more blocks in FIG. 3 may be performed using one or more components as described in FIGS. 1-2 and 4. While the various blocks in FIG. 3 are presented and described sequentially, one of ordinary skill in the art will appreciate that some or all of the blocks may be executed in different orders, may be combined or omitted, and some or all of the blocks may be executed in parallel. Furthermore, the blocks may be performed actively or passively.

As such, in accordance with one or more embodiments a concentration of a noxious gas is periodically measured with a first sensor (Step 361) and a level of ambient noise is periodically measured with a second sensor (Step 363). In accordance with at least one illustrative example, the first and second sensors may correspond to those described and illustrated with respect to FIG. 2 and indicated at 246 and 248, respectively.

In accordance with one or more embodiments periodic measurements of the concentration of the noxious gas and the ambient noise level are received from the first and second sensors (Step 365). Based on the received periodic measurements, a calculation is made for a cumulative total exposure to each of: the noxious gas and the ambient noise (Step 367). Based on the calculation of the cumulative total exposures, a calculation is made of a remaining permissible exposure time for each of: the noxious gas and the ambient noise (Step 369). Based on the calculation of the remaining permissible exposure times, a signal is transmitted to a first alarm, to activate the first alarm (Step 371). In accordance with at least one illustrative example, Steps 365, 367, 369 and 371 and 367 may be carried out by the first control/monitoring unit 249 and/or the second control/monitoring unit 251 described and illustrated with respect to FIG. 2. Further, the first alarm may correspond to that described and illustrated with respect to FIG. 2, and indicated at 252.

FIG. 4 schematically illustrates a computing device and related components, in accordance with one or more embodiments. As such, FIG. 4 generally depicts a block diagram of a computer system 485 used to provide computational functionalities associated with described algorithms, methods, functions, processes, flows, and procedures as described in this disclosure, according to one or more embodiments. In this respect, computer 485 may interface with a second control/monitoring unit 251 such as that described and illustrated with respect to FIG. 2, either directly (e.g., via hard-wired connection) or over an internal or external network 499. Alternatively, the computer 485 illustrated in FIG. 4 may correspond directly to the second control/monitoring unit 251 described and illustrated with respect to FIG. 2.

In accordance with one or more embodiments, the illustrated computer 485 is intended to encompass any computing device such as a server, desktop computer, laptop/notebook computer, wireless data port, smart phone, personal data assistant (PDA), tablet computing device, one or more processors within these devices, or any other suitable processing device, including both physical or virtual instances (or both) of the computing device. Additionally, the computer 485 may include a computer that includes an input device, such as a keypad, keyboard, touch screen, or other device that can accept user information, and an output device that conveys information associated with the operation of the computer 485, including digital data, visual, or audio information (or a combination of information), or a GUI.

The computer 485 can serve in a role as a client, network component, a server, a database or other persistency, or any other component (or a combination of roles) of a computer system for performing the subject matter described in the instant disclosure. The illustrated computer 485 is communicably coupled with a network 499. In some implementations, one or more components of the computer 485 may be configured to operate within environments, including cloud-computing-based, local, global, or other environment (or a combination of environments).

At a high level, the computer 485 is an electronic computing device operable to receive, transmit, process, store, or manage data and information associated with the described subject matter. According to some implementations, the computer 485 may also include or be communicably coupled with an application server, e-mail server, web server, caching server, streaming data server, business intelligence (BI) server, or other server (or a combination of servers).

The computer 485 can receive requests over network 499 from a client application (for example, executing on another computer 485) and responding to the received requests by processing the said requests in an appropriate software application. In addition, requests may also be sent to the computer 485 from internal users (for example, from a command console or by other appropriate access method), external or third-parties, other automated applications, as well as any other appropriate entities, individuals, systems, or computers.

Each of the components of the computer 485 can communicate using a system bus 487. In some implementations, any or all of the components of the computer 485, both hardware or software (or a combination of hardware and software), may interface with each other or the interface 489 (or a combination of both) over the system bus 487 using an application programming interface (API) 495 or a service layer 497 (or a combination of the API 495 and service layer 497. The API 495 may include specifications for routines, data structures, and object classes. The API 495 may be either computer-language independent or dependent and refer to a complete interface, a single function, or even a set of APIs. The service layer 497 provides software services to the computer 485 or other components (whether or not illustrated) that are communicably coupled to the computer 485. The functionality of the computer 485 may be accessible for all service consumers using this service layer. Software services, such as those provided by the service layer 497, provide reusable, defined business functionalities through a defined interface. For example, the interface may be software written in JAVA, C++, or other suitable language providing data in extensible markup language (XML) format or another suitable format. While illustrated as an integrated component of the computer 485, alternative implementations may illustrate the API 495 or the service layer 497 as stand-alone components in relation to other components of the computer 485 or other components (whether or not illustrated) that are communicably coupled to the computer 485. Moreover, any or all parts of the API 495 or the service layer 497 may be implemented as child or sub-modules of another software module, enterprise application, or hardware module without departing from the scope of this disclosure.

The computer 485 includes an interface 489. Although illustrated as a single interface 489 in FIG. 4, two or more interfaces 489 may be used according to particular needs, desires, or particular implementations of the computer 485. The interface 489 is used by the computer 485 for communicating with other systems in a distributed environment that are connected to the network 499. Generally, the interface 489 includes logic encoded in software or hardware (or a combination of software and hardware) and operable to communicate with the network 499. More specifically, the interface 489 may include software supporting one or more communication protocols associated with communications such that the network 499 or interface's hardware is operable to communicate physical signals within and outside of the illustrated computer 485.

The computer 485 includes at least one computer processor 491. Although illustrated as a single computer processor 491 in FIG. 4, two or more processors may be used according to particular needs, desires, or particular implementations of the computer 485. Generally, the computer processor 491 executes instructions and manipulates data to perform the operations of the computer 485 and any algorithms, methods, functions, processes, flows, and procedures as described in the instant disclosure.

The computer 485 also includes a memory 492 that holds data for the computer 485 or other components (or a combination of both) that can be connected to the network 499. For example, memory 492 can be a database storing data consistent with this disclosure. Although illustrated as a single memory 492 in FIG. 4, two or more memories may be used according to particular needs, desires, or particular implementations of the computer 485 and the described functionality. While memory 492 is illustrated as an integral component of the computer 485, in alternative implementations, memory 492 can be external to the computer 485.

The application 493 is an algorithmic software engine providing functionality according to particular needs, desires, or particular implementations of the computer 485, particularly with respect to functionality described in this disclosure. For example, application 493 can serve as one or more components, modules, applications, etc. Further, although illustrated as a single application 493, the application 493 may be implemented as multiple applications 493 on the computer 485. In addition, although illustrated as integral to the computer 485, in alternative implementations, the application 493 can be external to the computer 485.

There may be any number of computers 485 associated with, or external to, a computer system containing computer 485, wherein each computer 485 communicates over network 499. Further, the term “client,” “user,” and other appropriate terminology may be used interchangeably as appropriate without departing from the scope of this disclosure. Moreover, this disclosure contemplates that many users may use one computer 485, or that one user may use multiple computers 485.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims.

What is claimed is:

1. A system comprising:
a first sensor configured to periodically measure a concentration of a noxious gas;
a second sensor configured to periodically measure a level of ambient noise;
a first control/monitoring unit in communication with the first and second sensors, and configured to receive from the first and second sensors periodic measurements of the concentration of the noxious gas and the ambient noise level; and
a first alarm in communication with the first control/monitoring unit;
the first control/monitoring unit further being configured to:
based on the received periodic measurements, calculate a cumulative total exposure to each of:
the noxious gas; and
the ambient noise;
based on the calculation of the cumulative total exposures, calculate a remaining permissible exposure time for each of:
the noxious gas; and
the ambient noise; and
based on the calculation of the remaining permissible exposure times, transmit a signal to the first alarm to activate the first alarm.

2. The system according to claim 1, wherein the first control/monitoring unit is configured to transmit the signal when one or more predetermined thresholds, based on the calculation of the remaining permissible exposure times, are reached for either or both of:
the noxious gas; and
the ambient noise.

3. The system according to claim 2, wherein the one or more predetermined thresholds include a predetermined discrete time interval prior to a maximum permissible exposure time, for either or both of:
the noxious gas; and
the ambient noise.

4. The system according to claim 2, wherein the one or more predetermined thresholds include a predetermined proportion, less than 100 percent, of a maximum permissible exposure time for either or both of:
the noxious gas; and
the ambient noise.

5. The system according to claim 4, wherein the one or more predetermined thresholds additionally include a predetermined discrete time interval prior to the maximum permissible exposure time, for either or both of:
the noxious gas; and
the ambient noise.

6. The system according to claim 2, further comprising:
a second control/monitoring unit in communication with the first and second sensors, and configured to receive from the first and second sensors periodic measurements of the concentration of the noxious gas and the ambient noise level; and
a second alarm in communication with the first control/monitoring unit,
wherein the first control/monitoring unit is configured to transmit a signal to either or both of the first alarm and the second alarm, to activate either or both of the first alarm and the second alarm, when a maximum permissible exposure time has been reached for either or both of:
the noxious gas; and
the ambient noise.

7. The system according to claim 6, further comprising:
a second control/monitoring unit in communication with the first control/monitoring unit and with the second alarm,
wherein the first control/monitoring unit is configured to transmit a signal to the second control/monitoring unit to activate the second alarm, when the maximum permissible exposure time has been reached for either or both of:
the noxious gas; and
the ambient noise; and
wherein the second alarm is in a different location than the first alarm and the second control/monitoring unit is in a different location than the first control/monitoring unit.

8. The system according to claim 6, further comprising:
a portable sensing unit that includes one or more of the first and second sensors,
wherein the portable sensing unit includes the first alarm but not the second alarm.

9. The system according to claim 1, wherein the first control/monitoring unit is configured to:
determine at least two thresholds, based on the calculation of the remaining permissible exposure times, for each of:
the noxious gas; and
the ambient noise; and
transmit the signal when an earliest one of the determined thresholds is reached.

10. The system according to claim 1, further comprising a portable sensing unit that includes one or more of the first and second sensors.

11. The system according to claim 10, wherein the portable sensing unit further includes the first alarm and the first control/monitoring unit.

12. The system according to claim 10, wherein the portable sensing unit includes both of the first and second sensors.

13. The system according to claim 1, wherein the noxious gas is hydrogen sulfide.

14. A method comprising:
periodically measuring a concentration of a noxious gas, with a first sensor;
periodically measuring a level of ambient noise, with a second sensor;
receiving from the first and second sensors periodic measurements of the concentration of the noxious gas and the ambient noise level;
based on the received periodic measurements, calculating a cumulative total exposure to each of:
the noxious gas; and
the ambient noise;
based on the calculation of the cumulative total exposures, calculating a remaining permissible exposure time for each of:
the noxious gas; and
the ambient noise; and
based on the calculation of the remaining permissible exposure times, transmitting a signal to a first alarm to activate the first alarm.

15. The method according to claim 14, wherein the signal is transmitted when one or more predetermined thresholds, based on the calculation of the remaining permissible exposure times, are reached for either or both of:
the noxious gas; and
the ambient noise.

16. The method according to claim 15, wherein the one or more predetermined thresholds include a predetermined discrete time interval prior to a maximum permissible exposure time, for either or both of:
the noxious gas; and
the ambient noise.

17. The method according to claim 15, wherein the one or more predetermined thresholds include a predetermined proportion, less than 100 percent, of a maximum permissible exposure time for either or both of:
the noxious gas; and
the ambient noise.

18. The method according to claim 15, further comprising transmitting a signal to either or both of the first alarm and a second alarm, to activate either or both of the first alarm and the second alarm, when a maximum permissible exposure time has been reached for either or both of:
the noxious gas; and
the ambient noise.

19. The method according to claim 18, wherein:
one or more of the first and second sensors are disposed on a portable sensing unit; and
the portable sensing unit includes the first alarm but not the second alarm.

20. The method according to claim 14, further comprising:
determining at least two thresholds, based on the calculation of the remaining permissible exposure times, for each of:
the noxious gas; and
the ambient noise;
wherein the signal is transmitted when an earliest one of the determined thresholds is reached.
